(19) United States
(12) Patent Application Publication
(10) Pub. No.: US 2011/0284413 A1
(43) Pub. Date: Nov. 24, 2011

(54) BLOOD PLASMA CONTAINER

(75) Inventor: Heinz Meise, Schalksmuehle (DE)

(73) Assignee: Heinz Meise GmbH, Schalksmuehle (DE)

(21) Appl. No.: 13/138,277

(22) PCT Filed: Nov. 3, 2009
(86) PCT No.: PCT/EP2009/007846

§ 371 Date: Aug. 9, 2011

(30) Foreign Application Priority Data
Jan. 28, 2009 (DE) 20 2009 001 068.6

(51) Int. Cl. A61B 19/00

(52) U.S. Cl. 206/438

(57) ABSTRACT

The invention relates to a blood plasma container made of plastic, with a body that has a neck on one side, the body essentially having a rectangular horizontal cross section. Inside the body (1) a center link (1a) has been provided. The invention also relates to a blood plasma container made of plastic, with a body that has a neck on one side, the body essentially having a rectangular horizontal cross section. At least one holder (19) for sampling tubes (20) has been provided.

The invention relates to a blood plasma container made of plastic, with a body that has a neck on one side, the body essentially having a rectangular horizontal cross section.

Blood has numerous functions in a human body. It has a transport function, for example, because with the bloodstream numerous dissolved substances as well as warmth are delivered from their place of production or intake to their place of decomposition or excretion. It also has a respiratory function, a nourishing and rinsing function and a defense function. Blood consists of cells, the red blood cells (erythrocytes), the white blood cells (leukocytes) as well as thrombocytes and the surrounding liquid, the blood plasma (Deetjen/Speckmann, “Physiologie”, Urban & Schwarzenberg publishing house, 1992, p. 261). So, blood plasma is a blood liquid that is free of cells. Plasma is a liquid that is rich in proteins. Their composition is quite similar to that of interstitial fluid (Deetjen/Speckmann, ibid., p. 265). Blood plasma is stored in containers, usually bags or bottles.

Blood plasma containers as described above have actually been well-known (cf. DE 20 2007 012 912 U1). They provide a reliable and space-saving means to store and transport blood plasma upon its collection. Their dimensions correspond with those of plasma bags, so blood plasma containers are compatible with the transport and storage facilities provided for plasma bags.

The blood plasma containers known as such meet all requirements. One disadvantage in the use of existing blood plasma containers, however, is their behavior when the plasma is frozen. It has been found that those containers will expand by ca. one seventh of their volume during the freezing process. This makes the containers bulge, so the place-saving storage of numerous plasma containers that is actually a given cannot be guaranteed. It has also been found that the sampling tubes used to collect the blood plasma cannot be transported in a convenient manner. This has led to the tubes being fastened onto the containers with rubber bands in the case of the conventional blood plasma containers, which negatively affects handling and storage as well as the transportation of such containers.

The present invention is supposed to remedy this situation. The invention is based on the task of improving blood plasma containers. With the invention this task is solved by inserting a center link in the body of the bottle on the one hand. This center link prevents the risk of containers bulging during the process of freezing the blood plasma. Rather, the blood plasma container equipped with this invention will maintain its original shape so that the blood plasma containers can be stored and transported in the available devices.

The invention also provides for at least one holder for sampling tubes coming with each bottle. This means that the blood plasma sampling tubes can be fastened directly on the containers without the need of rubber bands and similar devices. At the same time the use of such holders offers the opportunity to fasten the sampling tubes directly on the containers, while the available, conventional storage and transport devices can still be used.

The invention has been improved advantageously through providing for the holders in the area of the center link. The holders will preferably consist of indentations that correspond to the contours of the sampling tubes. This makes it possible to insert the sampling tubes into the indentations and to transport them with the containers without any need for additional devices. Since the tubes can be inserted into the indentations, they will not cause any protrusions, so the handling of the containers will not be impaired by including the tubes.

Other improvements and developments of the invention have been listed in the other subclaims. One example of how the container will be designed is shown in the illustration and described in detail below. The following details are shown:

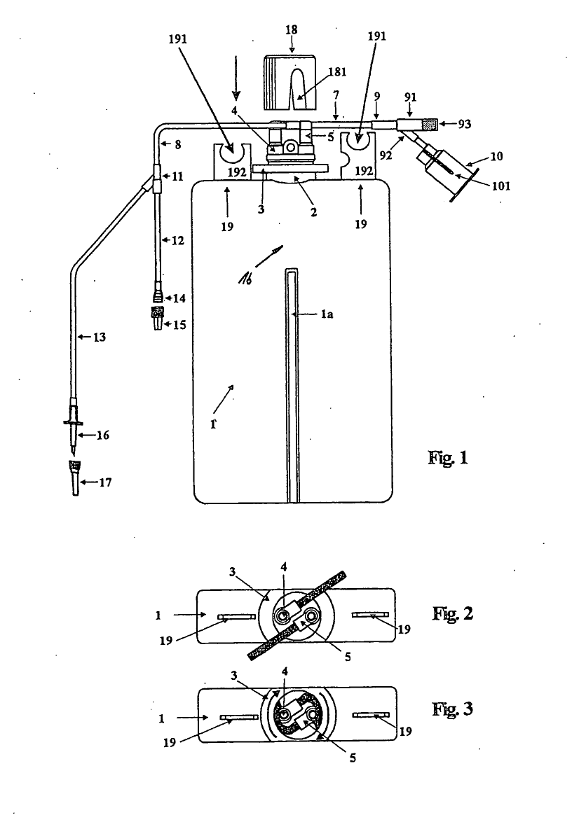

FIG. 1 View of a blood plasma container with closing cap, sampling, ventilation and testing units;

FIG. 2 Top view of the blood plasma container from FIG. 1,

FIG. 3 Top view of the blood plasma container from FIG. 1, with turned closing cap;

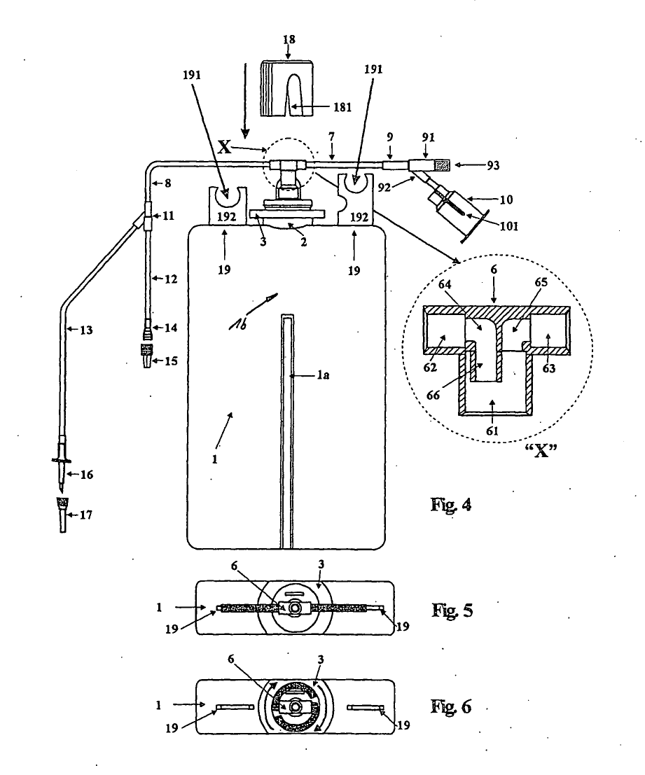

FIG. 4 View of a blood plasma container with closing cap, sampling, ventilation and testing units in a different design;

FIG. 5 Top view of the blood plasma container from FIG. 4;

FIG. 6 Top view of the blood plasma container from FIG. 4, with turned closing cap;

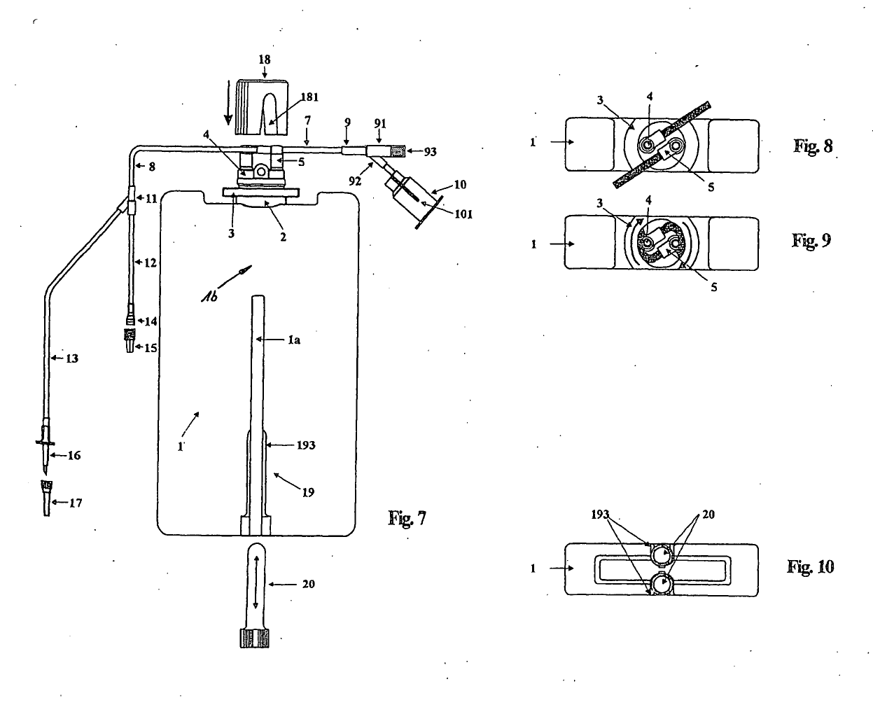

FIG. 7 View of a blood plasma container with closing cap, sampling, ventilation and testing units in a different design;

FIG. 8 Top view of the blood plasma container from FIG. 7;

FIG. 9 Top view of the blood plasma container from FIG. 7, with turned closing cap;

FIG. 10 Bottom view of the blood plasma container from FIG. 7;

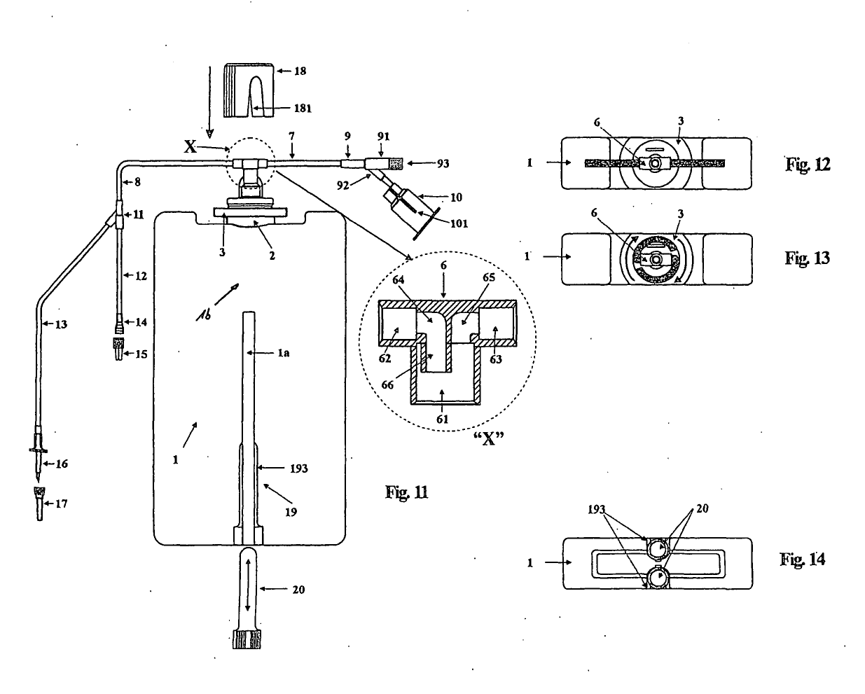

FIG. 11 View of a blood plasma container with closing cap, sampling, ventilation and testing units in another design;

FIG. 12 Top view of the blood plasma container from FIG. 11;

FIG. 13 Top view of the blood plasma container from FIG. 11, with turned closing cap, and;

FIG. 14 Bottom view of the blood plasma container from FIG. 11.

The blood plasma container used as a design sample was made of plastic. This plastic is polyethylene, preferably high-density polyethylene (HDPE). However, medium-density polyethylene (MDPE) or low-density polyethylene (LDPE) may be used as well. The choice of material is based on its meeting hygienic requirements. A sufficient stability of the container is an essential requirement, too, and the container has to remain stable when the plasma has been frozen.

The container consists of a body 1 that essentially has a rectangular horizontal cross section. It is provided with a neck 2 on one end. The neck 2 is closed with a container neck surface. The container neck surface 3 cannot be separated from the neck 2. Bottle 1, neck 2 and container neck surface 3 are shaped in one piece and manufactured in one production step.

The body 1 shows a thickness that corresponds to one third of the diameter of conventional plasma bottles. The dimensions of the container described in this invention are about the same as those of conventional plasma bags. This means that they have almost the same filling volume.

Inside body 1 a center link 1a will be provided. The center link 1a reaches from the bottom of the body 1 toward the neck 2. The center link 1a extends through ca. 70% of body 1. This causes a gap 1b to be formed between the center link 1a and the neck 2. The level of the center link corresponds to a filling level of ca. 650 ml blood plasma. Plasma containers are usually filled with 650 to 850 ml blood plasma, so this assures that the chambers on both sides of the center link 1a will be equally filled with blood plasma.

Also, the center link 1a basically creates a three-chamber system in the plasma container, i.e. one chamber on each side of the center link 1a and one chamber above the center link 1a on both sides of the gap 1b. This causes a substantially improved freezing behavior of the plasma. While a freezing time of ca. 2 to 2.25 hours is needed for plasma in a rectangular container without a center link, the container described in the invention will only require a freezing time of 1.5 to 2 hours under the same general conditions. This means that the plasma collected can be processed further much faster.

In the container neck surface 3 the design sample acc. to FIGS. 1 to 3 provides for two holes equipped with nozzles 5. The nozzles 5 cannot be separated from the container. They have been inserted close to the container neck surface 3. On the nozzles 4 fixtures 5 have been provided for a connection of hoses. The fixtures 5 in the design sample consist of L-shaped elbows that have been connected inseparably with the nozzles 4. The hoses have been connected inseparably with the ends of the fixtures 5 that are averted from the nozzles 4.

In the design sample acc. to FIGS. 4 through 6 only a hole has been provided for in the container neck surface, through which a connector 6 is inserted. Similar to the nozzles the connector 6 cannot be separated from the container. It has been inserted close to the container neck surface 3. The connector 6 has a T-shaped cross-section. The connector 6 has been provided with three mounting holes 61, 62, 63. The mounting hole 61 faces the neck 2 of the plasma container. The mounting holes 62 and 63 are set perpendicular to the mounting hole 61. They are diametrically opposed. The connections 64 and 65 extend from the mounting holes 62 and 63. They have been provided with a right angle so they lead to the mounting hole 61. For the connection 64 extending from the mounting hole 62 a tubular extension 66 has been provided that protrudes into the mounting hole 61.

The hoses connected to the fixtures 5 and the connector 6 are a ventilation hose 7 and a filling hose 8. The hoses 7 and 8 are made of plastics that can be sterilized.

The end of the ventilation hose 7 that is averted from the container is provided with a ventilation and testing unit. The ventilation and testing unit comprises a Y-piece that has been provided with a blocking device 91 and a branch-off 92. In the sample shown the blocking device 91 is a self-sealing filter. It is made of breathable, hydrophobic plastic, preferably polyethylene, and it shuts down on its own when there is a contact with fluids. As long as the self-sealing filter does not have any contact with fluids, it is breathable. It filters the air so a contamination of the blood plasma with germs from the air is prevented.

A coupling connection 10 is connected with the branch-off 92. A rubber needle valve 101 is integrated with it. The connection 10 serves to receive a vacuum sampling tube—not shown in the picture.

The end of the filling hose 8 that is averted from the container is provided with a collecting unit. This unit is provided with a switch 11 that is connected with two hoses 12 and 13 of different lengths. The end of hose 12 that is averted from the switch 11 is provided with a receptacle 14 and a protective cap 15. During the collection process the receptacle 14 is connected with a centrifugation cap. The end of hose 13 that is averted from the switch 11 is provided with a spike connector 16. A cap 17 can be put on the free end of this connector. The spike connector 16 is for extraction from bottles with physiological saline.

The nozzles 4 and the fixtures 5 acc. to the design sample in FIGS. 1 through 3 and the connector 6 acc. to the design sample in FIGS. 4 through 6 can be covered with a screw and closure cap 18. The cap has been provided with a device for locking into the neck 2 of the container. Also, the cap 18 has two slots 181 that extend from their end facing the container and axially to the end of the cap 18 that is averted from the container. The slots 181 are arranged in diametrical positions. They serve to put through the hoses 7 and 8. During the plasma donation process the cap 18 is in a position where the hoses 7 and 8 can be put easily through the slots 181. Upon completion of the plasma donation the hoses 7 and 8 are cut off with a thermal forceps and fused, as is well-known. The cap 18 is activated to fix the loose ends of the cut-off hoses 7 and 8. The cap 18 is turned for that purpose. During that process the loose ends of the hoses 7 and 8 are pulled into the cap 18 (FIGS. 3 and 6).

The body 1 is provided with at least one holder 19 for blood plasma sampling tubes 20. In the design sample acc. to FIGS. 1 through 6 one holder 19 has been inserted on each side of the neck 2. The holders 19 are constructed as a link 192. They provide for a clip-fastening of the sampling tubes. For that purpose the links 192 contain part-circular openings 191. The diameter of the openings is the same as the diameter of the sampling tubes. For fastening the tubes are pressed into the openings 191, in the well-known manner. Thus they are fastened reliably to the container that is part of the new invention.

In the design samples acc. to FIGS. 7 through 14 the holders 19 are provided in the area of the center link 1a. They consist of indentations 193 that essentially correspond to the contours of the sampling tubes 20. The sampling tubes 20 can be pushed into the indentations and removed again. When they are pushed into the holders the head of the sampling tubes 20 ends up level with the bottom of the plasma container.

### SUMMARY

The invention relates to a blood plasma container made of plastic, with a body that has a neck on one side, the body essentially having a rectangular horizontal cross section. Inside the body (1) a center link (1a) has been provided.

The invention also relates to a blood plasma container made of plastic, with a body that has a neck on one side, the body essentially having a rectangular horizontal cross section. At least one holder (19) for sampling tubes (20) has been provided.

1. Blood plasma container made of plastic, with a body that has a neck on one side, the body essentially having a rectangular horizontal cross section, wherein the container comprises a center link (1a) in the center of the body (1).

2. Blood plasma container according to claim 1, wherein the center link (1a) reaches from the bottom of the body (1) toward the neck 2.

3. Blood plasma container according to claim 1, further comprising a gap (1b) formed between the center link (1a) and the neck (2).

4. Blood plasma container made of plastic, with a body that has a neck on one side, the body essentially having a rectangular horizontal cross section, wherein the container further comprises at least one holder (19) for sampling tubes (20).

5. Blood plasma container according to claim 4, wherein a holder (19) is on each side of the neck (2)

6. Blood plasma container according to claim 4, wherein the holders (19) enable the sampling tubes to be clip-fastened.

7. Blood plasma container according to claim 4, wherein holders (19) are provided in the area of the center link (1a).

8. Blood plasma container according to claim 1, wherein the holders (19) comprise indentations (193) that essentially correspond to the contours of the sampling tubes (20).
